(19) United States
(12) Patent Application Publication
(10) Pub. No.: US 2025/0022611 A1
(43) Pub. Date: Jan. 16, 2025

(54) METHOD FOR OPTIMIZING A MEDICAL TRAINING PROCEDURE

(71) Applicants: Andrea LUCA, Milano (IT); OSPEDALE GALEAZZI S.P.A., Milano (IT)

(72) Inventor: Andrea LUCA, Milano (IT)

(73) Assignee: OSPEDALE GALEAZZI S.P.A., Milano (IT)

(21) Appl. No.: 18/711,458

(22) PCT Filed: Nov. 23, 2022
(86) PCT No.: PCT/EP2022/083033

§ 371 Date: May 17, 2024

(30) Foreign Application Priority Data
Nov. 26, 2021 (IT) 102021000029927

(51) Int. Cl. G16H 50/50; G06Q 10/0639

(52) U.S. Cl. CPC G16H 50/50; G06Q 10/06398

(57) ABSTRACT

A computer implemented method for optimizing a medical training procedure is disclosed using virtual reality and/or augmented reality on a virtual patient in a virtual or real operating room. The method includes assigning an initial specific competence level to the user before the training procedure, acquiring action data indicative of the virtual actions performed by the user, determining the presence of a deviated action, determining a final performance index of the user, and modifying or confirming the expected pathway of the training procedure based on the initial competence level of the user and the final performance index, and updating the expected pathway stored in the database if the expected pathway is modified.

The invention relates to a method for optimizing a medical training procedure, in particular a virtual reality or augmented reality procedure. The invention also relates to a corresponding workstation for carrying out said medical training procedure, in particular a virtual reality procedure. In addition, the invention relates to a computer program for implementing said method as well as a storage medium comprising said computer program.

In the past years, educational pathways in the medical field have been reorganized with the aim of optimizing the acquisition of competences and their assessment, so as to reduce the risks to both health care professionals and end users. Educational training in medicine is pressed in between two urgent needs: to ensure the uppermost level of safety for the patient, and to ensure the highest level of competence for health care professionals through an efficient, reproducible, and measurable transfer of expertise. Virtual reality (VR) simulations have been repeatedly tested, initially as a positive reinforcement for more traditional educational pathways and, more recently, as their potential substitute.

The application of Virtual Reality, Augmented Reality (AR) or Mixed Reality (MR) for medical trainings is becoming more and more popular and several simulations for different medical specialties are already available in the market. The most challenging aspect of the simulation is to recreate an immersive experience through an accurate reproduction of the environment (graphics and sounds) and a smooth mechanics of the human-devices-computer interface. VR application ranges from anatomic exploration of the human body, with different levels of complexity (guided tours, evaluation tests, interaction and manipulation of the anatomic structures), to the simulation of routine or highly complex medical procedures including propaedeutic training for robotic surgery.

It has been recently proven that a VR simulation can allow an authentic surgical experience providing an accurate human-machine interaction according to the user skills, for example for orthopaedic procedures.

However, a realistic threat that might affect the VR simulation is its repetitiveness (directly affecting the user's engagement). Without appropriate corrections, the user might experience multiple times precisely the same training pathway. Although the repetitiveness can be helpful for unskilled trainees (e.g. advanced beginners), allowing them to acquire the appropriate workflow, it might spoil the experience for more advanced trainees (competent and proficient), dramatically reducing the simulation durability.

Also, an inaccurate identification of the pre-training level of expertise of the user may lead to an unsatisfactory experience both for unskilled (advanced beginner) or advanced (competent and proficient) trainees. The first may go through an inappropriate learning process with a reduced transfer of knowledge (advanced beginners cannot perform correctly if not guided in the procedural workflow).

In addition, VR simulations are not always able to evaluate whether a deviation from an expected action is to be considered as a mistake of an unskilled user or a valuable alternative proposed by a proficient user.

Examples of the present disclosure seek to address or at least alleviate the above problems.

According to a first aspect of the invention there is provided a computer implemented method for optimizing a medical training procedure, in particular a virtual reality or augmented reality procedure, carried out by a user on a virtual patient in a virtual or real operating room, wherein the training procedure includes a sequence of consecutive expected actions defining an expected pathway stored in a database and the user performs a sequence of consecutive virtual actions corresponding to each of the expected actions during said training procedure, the method comprising:
assigning an initial specific competence level to the user among a plurality of competence levels by a competence module before starting the training procedure;
acquiring action data indicative of the virtual actions performed by the user during the training procedure;
determining the presence of a deviated action by a deviation module as a consequence of a deviation of at least one of the virtual actions performed by the user during the training procedure from the corresponding expected action;
determining a final performance index of the user based on at least the number of deviated actions during the training procedure by a performance module; and
modifying or confirming the expected pathway of the training procedure based on the initial competence level of the user and the final performance index, and updating the expected pathway stored in the database if the expected pathway is modified.

In a second aspect of the invention there is provided a computer program comprising computer readable instructions which, when implemented on a computer, causes the computer to carry out a method as defined above.

In a third aspect of the invention there is provided a storage medium comprising the computer program as defined above.

In a fourth aspect of the invention there is provided workstation for carrying out a medical training procedure, in particular a virtual reality procedure, the workstation comprising:
at least a computer system implementing computer readable instructions of the computer program product as defined above;
at least a head mounted device, in particular a virtual reality headset; and
at least a haptic device.

Other aspects and features are defined in the appended claims.

Examples of the disclosure may make it possible to optimize a medical training procedure by improving the durability and the accuracy of the procedure, for example of the VR simulation, thereby balancing the cost/benefit analysis upstream of a corresponding software development. In addition, examples of the disclosure may make it possible to correctly identify the user's level of expertise, thereby avoiding a frustrating or trivial training process.

Examples of the disclosure will now be described by way of example only with reference to the accompanying drawings, in which like references refer to like parts, and in which:

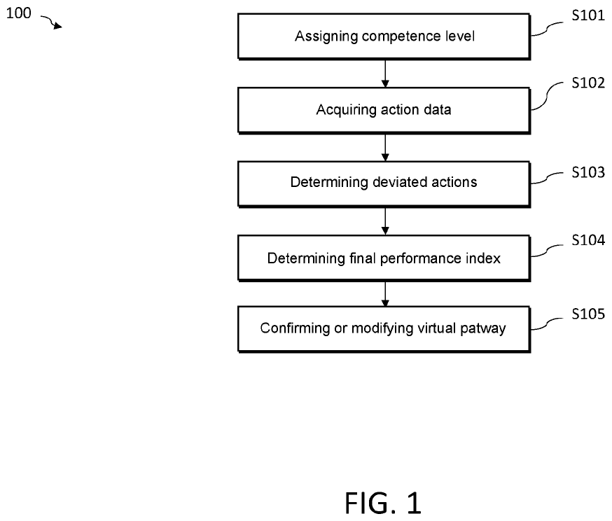

FIG. 1 shows a flowchart of the method for optimizing the medical training procedure according to an example.

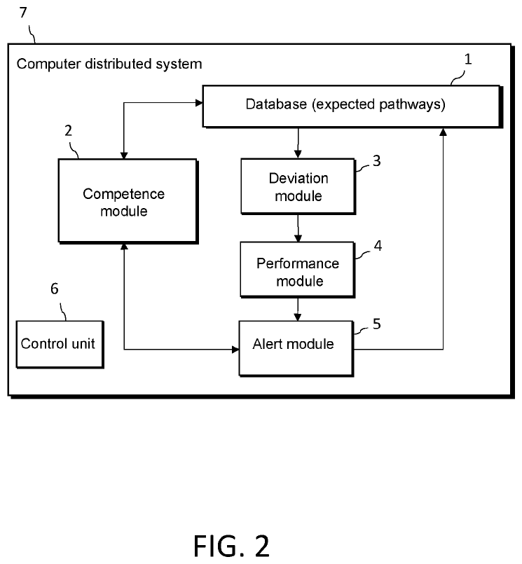

FIG. 2 shows a schematic representation of a computer distributed system according to an example.

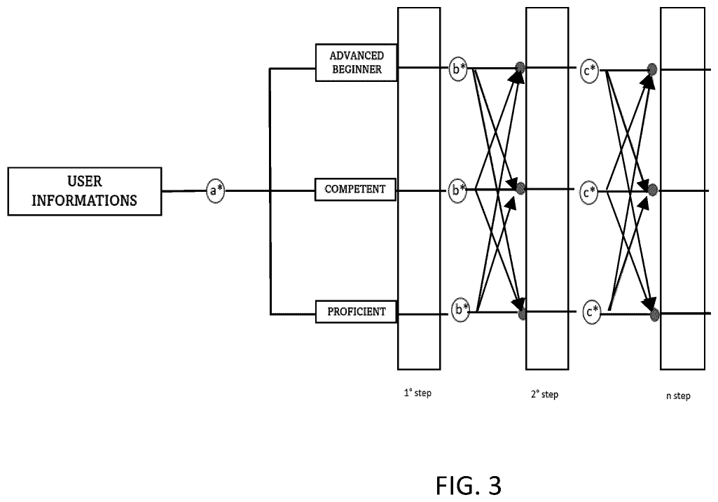

FIG. 3 shows a schematic representation of simulation patterns according to an example.

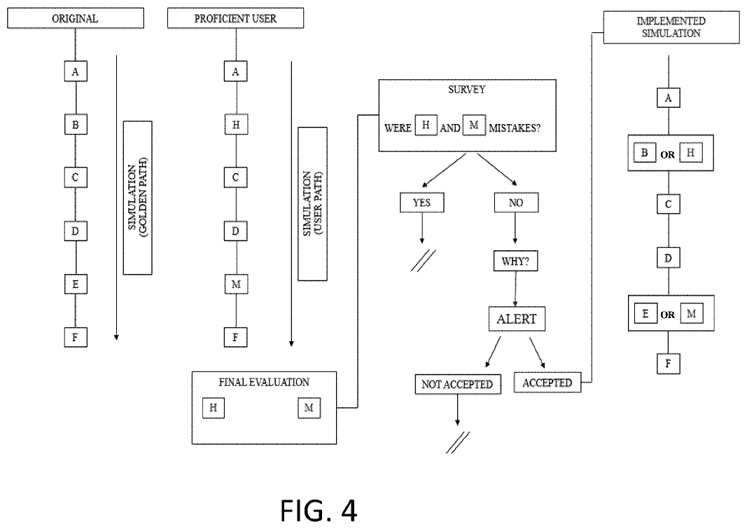

FIG. 4 shows a flow diagram of updating method steps according to an example.

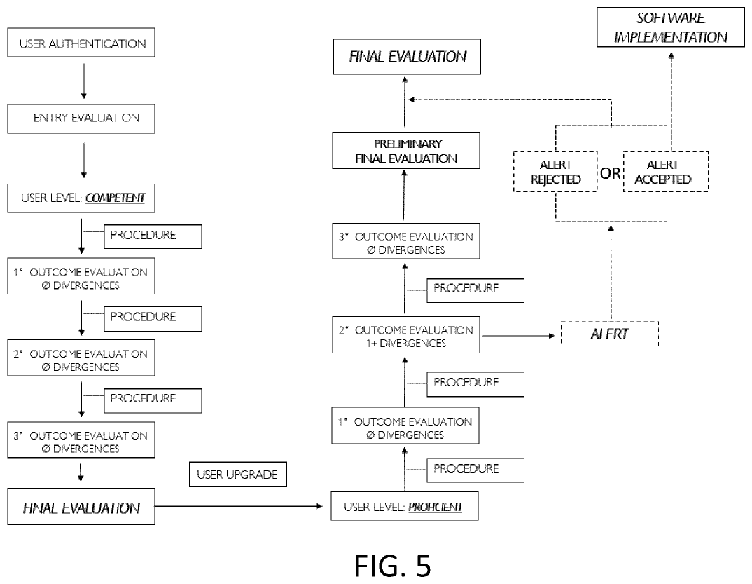

FIG. 5 shows a flow diagram of a specific example employing the method.

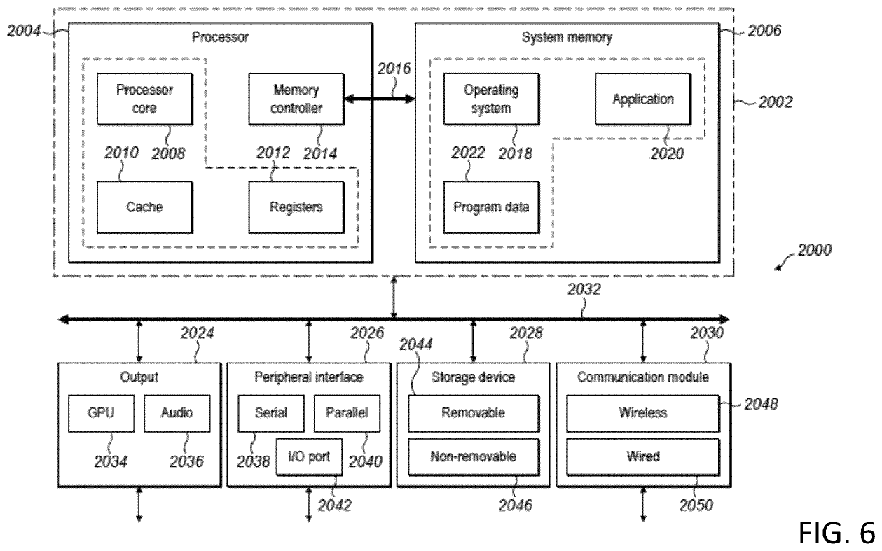

FIG. 6 shows a schematic representation of a computer system.

A computer implemented method for optimizing a medical training procedure is disclosed. In the following description, a number of specific details are presented in order to provide a thorough understanding of the examples of the disclosure. It will be apparent however to a person skilled in the art that these specific details need not be employed in order to practise the examples of the disclosure. Conversely, specific details known to the person skilled in the art are omitted for the purposes of clarity in presenting the examples.

FIG. 1 illustrates a method 100 for optimizing a medical training procedure. In particular, the method 100 refers to the optimization of a virtual reality procedure. It is noted that the same method 100 can also be applied to a procedure using an augmented reality (AR) technology or to a combination of VR and AR technology, i.e., mixed reality (MR) technology. The method is carried out by a user, i.e., a trainee, for example an inexperienced medical doctor at the beginning of his/her career or an expert medical doctor wishing to practice new approaches on particular surgical operations. The procedure is carried out on a virtual patient in a virtual operating room. For this purpose, suitable devices, such as haptic systems and head mounted devices (e.g. VR headsets) can be used. The training procedure includes a sequence of consecutive expected actions defining an expected pathway stored in a database. The consecutive expected actions are in particular expected information data that can be saved in the database. The user performs a sequence of consecutive virtual actions corresponding to each of the expected actions during said training procedure. The virtual actions of the user are indicated by virtual action data (i.e., action data) that can also be saved in the database. The virtual action data are the result of the application of the above-mentioned suitable devices.

It is noted that “operating room” is intended here as any type of room where a medical procedure can be carried out, for example an Operating Theatre, an Intensive care Unit, a first-aid station, a clinic, a doctor's office, an emergency room, a medical center, etc.

At step S101, the method 100 comprises assigning an initial specific competence level to the user among a plurality of competence levels before starting the training procedure. This can be performed by a competence module 2. For example, the plurality of competence levels can comprise at least a first level, a second level, and a third level based on at least the experience of the user in real medical procedures and on the number of virtual reality medical training procedures already carried out by the user. The first level can comprise advanced beginners, the second level can comprise competent users and the third level can comprise proficient users. Of course, a different number of levels can be considered. Also, sub-levels for each level can be taken into account. It is noted that the assignment of the initial specific competence level to the user is carried out by considering user data associated to the user (i.e., age, years of medical practice, years of experience in a particular medical field, past experience in using a particular medical tool, etc.). and comparing these user data with reference data. For example, a quick entry test can be carried out to assign the initial specific competence level.

At step S102, the method comprises acquiring action data indicative of the virtual actions performed by the user during the training procedure. Based on the activity of the user, for example using one or more dedicated haptic devices and/or visual images of the user, the virtual actions of the user can be evaluated and action data can be saved in the database. In other words, the acquisition of said action data serves to control and monitor the behaviour of the user during the medical training procedure.

Accordingly, the presence of a deviated action is determined at step S103 as a consequence of a deviation of at least one of the virtual actions performed by the user during the training procedure from the corresponding expected action. This can be performed by a deviation module 3, thereby comparing expected information data with (virtual) action data. It is noted that in order to avoid the continuous determination of a deviated action also in case the virtual action of the user differs in a very minimal way from the expected action, a tolerance range is present. This tolerance range can refer to a time value, to dimensions of a physical area, to coordinates of an object or of the user, etc. In other words, the tolerance range defines an acceptance error between a deviated action and an expected action so that if the deviated action is within said tolerance range it is considered as an expected action. For example, the correct positioning of one or more tools (e.g. fluoroscope, ventilator, carriage, surgical instruments, etc.) useful for the medical training procedure is not identified by a single isolated place of the virtual or real operating room but rather working areas are identified in the operating room, these working areas defining an accepted region where the tools are considered correctly positioned. In case the tool is positioned or is assigned outside said working area, a deviated action is determined. On the other hand, if the tool is positioned or is assigned inside said working area, it is considered a correct action and no deviated action is determined. In a similar way, the correct duration of an action is not identified by a precise time value but by a range of values. For example, if a single action is expected to last a value of time t (e.g. 1 minute), a deviation of Δt (e.g. 20 seconds) can still be accepted as correct action.

At step S104 a final performance index of the user based on at least the number of deviated actions during the training procedure is determined. This can be performed by a performance module 4. It is noted that the final performance index is determined at completed/finished training procedure. In other words, after the user has completed the training, the (virtual) action data performed till the end of the procedure are compared to the information data that were expected to be performed for the entire training procedure in order to determine the number of deviated action of the user (if any) throughout the completed training procedure.

Hence, the final performance index reflects the behaviour of the user in carrying out the completed training procedure. A high number of deviated actions is considered as a high number of errors from the user and therefore results in a negative final performance index. On the other hand, the absence or a very limited number of deviated actions is considered as a correct behaviour of the user and therefore results in a positive final performance index. The final performance index can be represented by a performance value varying within a performance range. For example, if said value is above a certain reference value (or final performance threshold), the final performance index can be considered positive, i.e., the user has correctly finished the training procedure without or almost without errors. On the other hand, if the performance value is below a certain reference value (or final performance threshold), the final performance index can be considered negative, i.e., the user has finished the training procedure making several errors. It is clear that this is just an example and the final performance index can be considered positive if the performance value is below a certain reference value (or final performance threshold) and can be considered negative if is above said reference value.

The expected pathway (i.e., the expected information data) of the training procedure is modified or confirmed at step S105 based on the initial competence level of the user and the final performance index, and the expected pathway stored in the database 1 is updated if the expected pathway is modified. In particular, the expected information data stored in the database 1 are updated if the expected information data are modified.

Accordingly, it is possible to increase the durability and reduce the repetitiveness of the procedure. Furthermore, the procedure can be self-corrected. As a matter of fact, the outcomes of specific users groups based on the competence levels can be analyzed in order to update and refine the simulation over time, thereby further improving the durability.

It is noted that the present method is basically directed to optimize the training procedure and not to assess the users skills. On the other hand, the skills of the user can contribute to optimize the training procedure, for example for other users. For this reason, the modification or confirmation of the expected pathway is based on the final performance index of the user (in addition to the initial competence level of the user), that is the performance index calculated after the user has finished the training procedure. As described in the following, an intermediate performance index can also be calculated before the user has finished the training procedure. However, this is optional and the final performance index is calculated anyway, without stopping the training procedure, or without disenrolling the user. The final performance index is used to optimize the training procedure carried out in a subsequent round of training procedure for the same user or for a different user, even if the performance index (final or intermediate) is above or below a specific performance threshold value. In other words, an optimization of the medical training procedure, i.e. a modification of the expected pathway, for example in terms of an update of this expected pathway, can only be successfully achieved if the user completely finishes the training procedure and a final performance index is calculated.

In one example, the method comprises comparing action data indicative of the virtual actions of the user with information data of the corresponding expected actions defining the expected pathway. A deviated action can be determined if at least one of the following event occurs:
one or more medical instruments used in the virtual action are different from those in the corresponding expected action; and/or
the sequence of virtual actions is different from the sequence of expected actions; and/or
the time duration of at least one virtual action is longer or shorter than a reference time duration for the corresponding expected action; and/or
the number of virtual actions is different from the number of expected actions; and/or one or more medical instruments or objects in the virtual operating room are positioned in a different place from an expected positioning place; and/or
the virtual patient is positioned by the user in a different position from an expected patient position; and/or
the difference between the action data indicative of a virtual action and the information data of the corresponding expected action is outside a tolerance range.

It is clear that the above mentioned events are just examples and are not limitative. Other events can be taken into account for determining a deviated action.

In examples, the method 100 further comprises assigning a relevance weight to each deviated action before determining the final performance index. In this way, some deviated actions, as for example listed above, can be more relevant compared to other deviated actions in order to assess the final performance index. For example, using a wrong surgical instrument could be more relevant—i.e., it could have a stronger impact in the determination of the final performance index—than exceeding the expected operation time. A relevant deviated action is intended here as an action contributing to the determination of the final performance index. Therefore, a deviated action with a high relevance weight would contribute more to the determination of the final performance index compared to a deviated action with a low relevance weight. For example, the relevance weight can be a factor ranging from 0 to 1 given to the corresponding deviated action. The final performance index is the sum of the deviated actions, each multiplied by the corresponding relevance weight, and divided by the number of deviated actions. For example, the wrong position of the user in the virtual or real operating room, in particular relative to the virtual patient, is a fundamental issue and can be assigned with a factor 1, whereas the wrong position of the carriage on which the surgical tools are located is less relevant and can be assigned with a factor 0.5.

In addition or in alternative, the method 100 can further comprise assigning a relevance weight to each deviated action based on the competence level of the user. Therefore, the same deviated action carried out by an advanced beginner can be more or less relevant compared to the same action carried out for example by a proficient.

In one example, the expected pathway of the training procedure can have at least a complexity degree based on the initial specific competence level assigned to the user. For example, for the first level, i.e., the advanced beginner, the procedure can provide an assisted training. For the second level, i.e., competent, the procedure can be in free run procedure. For the third level, i.e. proficient, procedure can provide unexpected situations or anatomical variants. In particular, for each initial specific competence level assigned to the user there could be a plurality of possible scenarios that could be selected random or based on a previous training procedure of the same user.

In examples, the method comprises modifying or confirming the competence level of the user based on the initial competence level and the final performance index. For example, if several deviated actions are determined after a training procedure of an advanced beginner, the competence level can be maintained for a successive training procedure of the same user in order to provide the user with a similar situation and teaching again the user how to correctly behave. If no or few relevant deviated actions are determined after a training procedure of an advanced beginner, the competence level of the user can be upgraded to a higher competence level, e.g. competent, in order to provide the user with a different and more challenging situation. On the other hand, if several deviated actions are determined after a training procedure of a competent user, the competence level can be downgraded to a lower level, e.g. advanced beginner, in order to provide the user with a different and less challenging situation.

In examples, the method 100 can further comprise modifying the consecutive expected actions if the final performance index is above a final performance threshold and the competence level of the user has been changed compared to the initial competence level. In particular, the sequence of expected actions can be modified in case the user correctly finished the training procedure, i.e. the user has reached a positive final performance index or the final performance index is above a final performance threshold, and the competence level is upgraded to a higher level. In this case the sequence of expected actions can be updated to the different level of competence.

In addition or in alternative, the method 100 can comprise modifying the consecutive expected actions if the final performance index is above a final performance threshold, the competence level of the user has been confirmed compared to the initial competence level, and the training procedure has been repeated by the user more than one time. In particular, the sequence of expected actions can be modified in case the user correctly finished the training procedure, i.e. the user has reached a positive final performance index or the final performance index is above a final performance threshold, the competence level is maintained at the initial level, and the user has repeated the procedure several times. In this way, the training procedure is improved since it is avoided that the user remembers the steps of the procedures already carried out in the previous procedure.

In examples, the method 100 further comprises saving a user history of the virtual actions if the competence level of the user has been changed compared to the initial competence level, wherein the expected pathway of the training procedure is confirmed or modified based also on the user history. In this way, the past behaviour of the user can be taken into account when proposing an expected pathway to him/her for a new training procedure. The final performance index of a user can be compared to his/her performance index carried out in a previous training procedure in order to evaluate the presence or absence of improvements. Also, the performance index of a user can be compared to an average performance index value of other users, preferably of the same competence level. In this way, it is possible to have an idea of the own actual competence compared to the average competence of other users (of the same competence level) carrying out the same training procedure.

If the final performance index is below a final performance threshold, the method 100 can further generate a final alert signal. This can be performed by an alert module 5. In particular, in case several deviated actions, or very relevant deviated actions are determined during the training procedure, i.e. the user has reached a negative final performance index or the final performance index is below a final performance threshold, a signal can be generated to inform the trainer and the user of a negative performance. Advantageously, a detailed report can be also generated showing the errors or deviated actions and eventually suggesting the correct corresponding actions. As mentioned above, the final performance index is a relative concept and can be considered negative (i.e., bad) or positive (i.e., good) if it is below or above a final performance threshold. In this particular example, the value of the final performance index decreases by increasing the number of deviated actions and is considered negative if below the threshold. In another analogous example, the value final performance index can increase by increasing the number of deviated actions and is considered positive if below the threshold.

According to an example, the method 100 can further comprise, based on the initial competence level of the user, modifying the training procedure by adding the deviated action in the expected pathway of the training procedure as alternative to the corresponding expected action. This is extremely useful if the user has a high competence level, i.e., proficient. In this case, the deviated action is not considered as an error but rather as an alternative to the expected action in the sequence provided by the training procedure. In an example, the user can send a feedback (for example to the computer system 7 or a dedicated person) requesting that the deviated action be considered as a possible alternative. The request can be evaluated in a validation step by the corresponding computer system 7 or the dedicated person in order to accept or not the deviated action as alternative to the expected action in the sequence provided by the training procedure. Advantageously, the computer system 7 can use a machine learning approach.

In addition or in alternative, the method 100 can further comprise, based on the initial competence level of the user, modifying the training procedure by replacing an expected action with a corresponding deviated action in the expected pathway of the training procedure. As mentioned above, this is useful if the user has a high competence level, i.e., proficient. In this case, the deviated action is not considered as an error but rather as an improvement to the expected action in the sequence provided by the training procedure. In an example, the user can send a feedback (for example to the computer system 7 or a dedicated person) requesting that the deviated action be considered as a possible improvement. The request can be evaluated in a validation step by the corresponding computer system 7 or the dedicated person in order to accept or not the deviated action as an improvement to the expected action in the sequence provided by the training procedure.

It is noted that in both above-mentioned cases (i.e., when the deviated action is added in the expected pathway as an alternative or when the deviated action is replacing an expected action in the pathway), the validation step can occur automatically, without a dedicated feedback from the user and/or without a supervision of an external person. For example, the validation step can occur automatically if that deviated action is repeated by the user (and registered) each time a training procedure is carried out by the user (or by different users, in particular with the same competence level). Advantageously, if the user has a high competence level, i.e., proficient, and repeats a deviated action in the expected pathway of the training procedure each time he/she carries out the training procedure, that a deviated action can be considered as an improvement and the training procedure is modified by adding the deviated action in the expected pathway of the training procedure as alternative to the corresponding expected action or the training procedure is modified by replacing an expected action with a corresponding deviated action in the expected pathway of the training procedure. For example, this can occur if the user repeats the deviated action N time, where N>3. It is noted that it is not necessary that the deviated action is repeated always by the same user. The validation step can also occur if the same deviated action is repeated and registered by different users (possibly having the same competence level) when carrying out a training procedure.

It is furthermore noted that the training procedure is modified by first adding the deviated as alternative to the corresponding expected action. In case N is greater than M (with M equal or greater than 5), or following a dedicated user feedback, the training procedure could then be modified by replacing the corresponding expected action with the deviated action.

In examples, the method 100 can further comprise determining at least an intermediate performance index of the user based at least on the number of deviated actions occurred during the training procedure up to an intermediate evaluation point of the expected pathway; and
assigning a different specific competence level, or confirming the competence level, to the user for the remaining duration of the training procedure based on a comparison between said intermediate performance index and an intermediate performance threshold.

In other words, the competence level of a user can be upgraded or downgraded during the training (i.e., even is the training procedure is not finished yet), if the user is correctly carrying out the training procedure or is strongly deviating from the expected pathway, respectively.

In order to provide the user with a constant feedback on his/her performance, the method can further comprise providing the user with an alert message each time a deviated action is determined during the training procedure. In this way, the attention of the user is increased during the training procedure.

FIG. 2 describes a computer distributed system 7 that can be employed to carry out the method 100 described above. The system 7 comprises a database 1 where a sequence of consecutive expected actions (i.e. expected information data) are stored. Each sequence of actions (i.e., each combination of expected information data) defines an expected pathway. The database 1 can comprise a plurality of expected pathways (i.e. a plurality of combined expected information data). The system also comprises a competence module 2 for assigning a specific initial competence level to the user and a deviation module 3 for determining the presence of a deviation of at least one of the virtual actions performed by the user during the training procedure from the corresponding expected action. Accordingly, a final performance index is determined by a performance module 4. The performance module 4 can also be used to generate an intermediate performance index based at least on the number of deviated actions occurred during the training procedure up to an intermediate evaluation point of the expected pathway. The system 7 additionally comprises an alert module 5 for generating an alert signal in case the final performance index is below a final performance threshold. A control unit 6 is present to control the several modules of the system 1. The alert module 5 can also be used to generate an alert message each time a deviated action is determined during the training procedure.

It is noted that the competence module 2 directly interacts with the database 1 in order to define the expected pathways based on the competence level of the user. The competence module 2 also directly interacts with the alert module 5 for the generation of the final alert signal and/or the alert message. As a matter of fact, the final alert signal and/or the alert message are generated based on the final performance index (and/or the intermediate performance index) and the competence level of the user. Based on the competence level of the user and the final (and/or intermediate) performance index, the expected pathway stored in the database 1 can be updated.

According to an example, the present method 100 for optimizing a training procedure can comprise five main steps. In a first main step the user is authenticated. New users select the username and password. The password may also be automatically generated. A registered user can access through standard authentication protocol.

In a second main step, the user is positioned. A pre-learning screening allows to assess the initial level of expertise or competence (performance-based assessment of expertise). Several aspects may help to define the level of expertise in a given field. The method evaluates some of these aspects considered to be more relevant for the training session:
Experience (number of years in practice);
Certification;
Peer acclamation;
Entry test (pre-existing knowledge);
Self-evaluation;
Number of previous completed trainings.

Each aspect impacts in a specific and given way on the pre-learning assessment.

As a result of the second main step, the trainee or user is included in the training, based on his/her level of expertise in one of the three groups: Advanced beginner, competent, and proficient. It is clear that additional groups or sub-groups can be taken into account, wherein the complexity of the simulated experience can differ in the three groups.

In the third main step, the user starts the training.

The user should carry out specific tasks (e.g.: to organise the objects in the surgical room, to define the patient positioning, to select the appropriate surgical tool). The user's performance are compared with the “correct” procedure (named: golden path that is the way to perform the procedure according to the original developer that by definition is proficient).

According to the complexity of the procedure, the training includes at least two evaluation points (in FIG. 3 named gates). At these stages, based on the number of incorrect tasks made by the user (outcome measurement), the competence level can possibly be modified (beginner may upgrade, competent may upgrade or downgrade, or proficient may downgrade). Accordingly, the user can change the pathway during the training procedure. For users who complete the training more than once (multiple logins) different patterns of simulation can be provided subject to the features of the simulated procedure.

FIG. 3 illustrates a preliminary data analysis of the user at point a*, a first step analysis at point b* and a second step analysis at step c*. Of course, an increased number of step analysis can be considered during the training procedure. This method positively impacts both on increasing the durability and on reducing the repetitiveness of the training procedure.

In a fourth main step a final evaluation is carried out. In particular, a global performance is evaluated by assigning a score (outcome measurements: quantitative analysis). In addition, information regarding the nature of the incorrect tasks and their explanations according to the developer technique is provided (qualitative analysis).

For users who complete the training more than once (multiple logins) a performance index can be calculated by exploring both discrimination ability and consistency of the user. The index may be used to evaluate the final level of expertise or competence for each user. Different types of performance indexes may be used, for example the Cochrane-Weiss-Shanteau index (CWS index).

In a fifth main step, a self-implementation is carried out. The same medical training procedure might be carried out by different experts in different ways (there might be slight differences in the way experts perform the given medical procedure). This happens very often in medicine.

The method 100 can take this aspect into account. In particular, variations from the original/expected technique (designed by the developer of the simulation) are accepted when coming from peers (experts that are considered to have the same level of expertise of the developer) and are not considered as mistakes. If, at the beginning of the procedure, the user is classified as proficient, potential incorrect tasks may be identified. During the fourth main step (final evaluation), the user can be asked to answer a survey (possible questions may relate why the incorrect tasks were done, and if they were done by purpose, what the proficient user think is the advantage when compared with what was designed by the developer). The survey can generate an alert that will be reviewed by the developer. If the developer accepts the proposed variations, they will be included in the simulation enabling its implementation (semi-automatic). This step can also be carried out automatically by the computer distributed system 7, for example if the deviation from the original/expected action is repeated more than one time by a single user or by different users when repeating the same training procedure.

The higher the number of proficient users using the training procedure, the higher the possibility to include in the expected pathways all the potential technical nuances of the procedure. These aspects are clearly illustrated in FIG. 4. Compared to the original sequence of expected actions (golden path), the proficient user employs an action H instead of the expected action B and an action M instead of an expected action E. After a final evaluation of the training procedure, a survey is carried out (automatically or semi-automatically). If the actions H and M are mistakes, the procedure ends with a final performance index and a final report. If the actions H and M are mistakes, an alert signal is generated and the steps can be accepted in the training procedure as alternative or as substitutive steps based on the fact that the steps H and M could provide an objective advantage compared to the expected steps B and E, respectively. Accordingly, the expected pathway is updated and stored in the database 1.

In the following, a specific example for a surgical access to the thoracic spine and spinal fixation with pedicle screws is described in combination with FIG. 5.

The user accesses the procedure through a preliminary authentication.

Once the authentication is carried out, the user must complete the entry evaluation that, combined with the previous step, defines his/her level of expertise: in this example, the user's expertise falls into the category “competent” (second level).

The user performs the simulation, and considering his/her level of expertise (“competent”), the software will not provide any suggestions nor explanations of the procedure.

The user interactions with the simulated environment (virtual action data) are recorded and compared with the original pre-set workflow designed in advance (expected information data).

The user carries the simulation out without significant deviations or divergences from the original pre-set workflow, thus upgrading, after the final evaluation, his/her level of expertise to “proficient” (third level).

The user performs the simulation again, and considering his/her new level of expertise (“proficient”), unexpected scenarios (e.g., anatomic variants or complications) can be provided.

For example, the unexpected event of a “pedicle fracture” can be generated, a condition that, according to the pre-set workflow, has two potential solutions:
1. To implant the screw anyway; or
2. To use a different vertebral fixation systems (e.g. sub-laminar bands).

The proficient user decides not to insert the screw, instrumenting the adjacent vertebras. According to the original workflow, this decision falls into the category “mistakes”; however, considering the user's level of expertise, an alert is generated that will be recorded and analyzed automatically by the computer system 7 or by a dedicated person.

Although not initially included in the potential solutions, the user action (i.e., not to insert the screw, instrumenting the adjacent vertebras) may represent an adequate option to manage the unexpected situation (pedicle fracture). Therefore, deviated action is accepted. For example, the deviated action can be accepted automatically because the user has repeated the deviated action each time he/she carries out the training procedure (i.e. more than 3 times or more than 5 times).

The acceptance of the new solution has two main effects:
1. The final evaluation of the performance will not be affected (the user's action is no longer considered a mistake);
2. The implementation of the pathway: the event of “pedicle fracture” now has three potential solutions, the first two designed in advance and the latter suggested by the user.

It is clear that this method can be advantageously used also in different medical fields, e.g. cardiac surgery, surgery of the hand, training of nurses or operating room assistants, etc. The method can be structured as a program library of simulations covering several medical disciplines, wherein in each discipline several simulations of different procedures and variants are foreseen based on the competence level of the user, as described above.

The method can also be used for teaching specific chirurgical techniques or for showing the positioning and the installation of a medical device, such as prosthesis, osteosyntheses means, heart valve, external fixator, cardiovascular stent, etc. The medical device can be a generic device or a specific device reproducing all the 3D characteristics of a device of a particular factory, in case the aim is to teach the use of a specific type of a device.

FIG. 6 schematically shows a computer system for implementing methods and techniques of examples of the disclosure. In particular, FIG. 5 shows an example of a computing device 2000 for example which may be arranged to implement one or more of the examples of the methods described herein. In examples, the computing device 2000 comprises main unit 2002. The main unit 2002 may comprise a processor 2004 and a system memory 2006. In examples, the processor 2004 may comprise a processor core 2008, a cache 2010, and one or more registers 2012. In examples, the processor core 2008 may comprise one or more processing cores and may comprise a plurality of cores which may run a plurality of threads. The processor 2004 may be of any suitable type such as microcontroller, microprocessor, digital signal processor or a combination of these, although it will be appreciated that other types of processor may be used.

In examples, the processor core 2008 may comprise one or more processing units. In examples, the processor core 2008 comprises one or more of a floating point unit, an arithmetic unit, a digital signal processing unit, or a combination of these and/or plurality of other processing units, although it will be appreciated that other processing units could be used. In examples, the cache 2010 may comprise a plurality of caches such as a level one cache and a level two cache, although other appropriate cache arrangements could be used.

In examples, the processor 2004 comprises a memory controller 2014 operable to allow communication between the processor 2004 and the system memory 2006 via a memory bus 2016. The memory controller 2014 may be implemented as an integral part of the processor 2004, or it may be implemented as separate component.

In examples, the system memory 2006 may be of any suitable type such as non-volatile memory (e.g. flash memory or read only memory), volatile memory (such as random access memory (RAM)), and/or a combination of volatile and non-volatile memory. In examples, the system memory 2006 may be arranged to store code for execution by the processor 2004 and/or data related to the execution. For example, the system memory may store operating system code 2018, application code 2020, and program data 2022. In examples, the application code 2020 may comprise code to implement one or more of the example methods described herein, for examples to implement the steps described above with reference to FIGS. 1 and 4. The application code 2020 may be arranged to cooperate with the program data 2022 or other media for example to allow the train of the cognitive and visuo-spatial path generating model.

In examples, the computing device 2000 may have additional features, functionality or interfaces. For example main unit 2002 may cooperate with one or more peripheral devices for example to implement the methods described herein. In examples, the computing device 2000 comprises, as peripheral devices, an output interface 2024, a peripheral interface 2026, a storage device 208, and a communication module 2030. In examples, the computing device comprises an interface bus 2032 arranged to facilitate communication between the main unit 2002 and the peripheral devices.

In examples, the output device 2024 may comprise output devices such as a graphical processing unit (GPU) 2034 and audio output unit 2036 for example arranged to be able to communicate with external devices such as a display, and/or loudspeaker, via one or more suitable ports such as audio/video (A/V) port. In examples, the peripheral interface 2026 may comprise a serial interface 2038, a parallel interface 2040, and a input/output port(s) 2042 which may be operable to cooperate with the main unit 2002 to allow communication with one or more external input and/or output devices via the I/O port 2042. For example, the I/O port 2042 may communication with one or more input devices such as a keyboard, mouse, touch pad, voice input device, scanner, imaging capturing device, video camera, and the like, and/or with one or more output devices such as a 2D printer (e.g. paper printer), or 3D printer, or other suitable output device. For example, signals may be received via the I/O port 2042 and/or the communication module 2030.

In examples, the storage device may comprise removable storage media 2044 and/or non-removable storage media 2046. For example, the removable storage media may be random access memory (RAM), electrically erasable programmable read only memory (EEPROM), read only memory (ROM) flash memory, or other memory technology, optical storage media such as compact disc (CD) digital versatile disc (DVD) or other optical storage media, magnetic storage media such as floppy disc, magnetic tape, or other magnetic storage media. However, it will be appreciated that any suitable type of removable storage media could be used. Non-removable storage media 2046 may comprise a magnetic storage media such as a hard disk drive, or solid state hard drive, or other suitable media, although it will be appreciated that any suitable non-removable storage media could be used. The storage device 2028 may allow access by the main unit 2002 for example to implement the methods described herein.

In examples, the communication module may comprise a wireless communication module 2048 and a wired communication module 2050. For example, the wireless communication module may be arranged to communicate wirelessly via a suitable wireless communication standard for example relating to wifi, Bluetooth, near field communication, optical communication (such as infrared), acoustic communication, or via a suitable mobile telecommunications standard. The wired communication module may allow communication via a wired or optical link for example by Ethernet or optical cable. However, it will be appreciated that any suitable communication module could be used.

Referring to FIG. 2, the competence module, the deviation module, the performance module, or the alert module may for example be implemented by the processor. In examples, one or more of the competence, and/or deviation, and/or performance, and/or alert module may be implemented by the main unit 2002, although it will be appreciated that other suitable implementations could be used. In examples, the training or analysis module may be implemented by the main unit 2002 in cooperation with the output device 2024, although it will be appreciated that other suitable implementations could be used.

It will be appreciated that in examples of the disclosure, elements of the disclosed methods may be implemented in a computing device in any suitable manner. For example, a conventional computing device may be adapted to perform one or more of the methods described herein by programming/adapting one or more processors of the computing device. As such, in examples, the programming/adapting may be implemented in the form of a computer program product comprising computer implementable instructions stored on a data carrier and/or carried by a signal bearing medium, such as floppy disk, hard disk, optical disk, solid state drive, flash memory, programmable read only memory (PROM), random access memory (RAM), or any combination of these or other storage media or signal bearing medium, or transmitted via a network such as a wireless network, Ethernet, the internet, or any other combination of these or other networks.

In other words, in examples, a computer program may comprise computer readable instructions which, when implemented on a computing device, cause the computing device to carry out a method according examples of the disclosure. In examples, a storage medium may comprise the computer program, for example, as mentioned above. It will also be appreciated that other suitable computer architectures could be used such as those based on one or more parallel processors. Furthermore, at least some processing may be implemented on one or more graphical processing units (GPUs). Although computing device 2000 is described as a general purpose computing device, it will be appreciated that this could be implemented in any appropriate device, such as mobile phone, smart phone, camera, video camera, tablet device, server device, etc., with modifications and/or adaptation if appropriate to the features described above, for example dependent on the desired functionality and hardware features.

Although a variety of techniques and examples of such techniques have been described herein, these are provided by way of example only and many variations and modifications on such examples will be apparent to the skilled person and fall within the spirit and scope of the present invention, which is defined by the appended claims and their equivalents.

1. A computer implemented method for optimizing a medical training procedure, in particular a virtual reality and/or augmented reality procedure, carried out by a user on a virtual patient in a virtual or real operating room, wherein the training procedure includes a sequence of consecutive expected actions defining an expected pathway stored in a database and the user performs a sequence of consecutive virtual actions corresponding to each of the expected actions during said training procedure, the method comprising:
assigning an initial specific competence level to the user among a plurality of competence levels by a competence module before starting the training procedure;
acquiring action data indicative of the virtual actions performed by the user during the training procedure;
determining the presence of a deviated action by a deviation module as a consequence of a deviation of at least one of the virtual actions performed by the user during the training procedure from the corresponding expected action;
determining a final performance index of the user based on at least the number of deviated actions during the training procedure by a performance module; and
modifying or confirming the expected pathway of the training procedure based on the initial competence level of the user and the final performance index, and updating the expected pathway stored in the database if the expected pathway is modified.

2. The method according to claim 1, wherein the method comprises comparing action data indicative of the virtual actions with information data of the corresponding expected actions, a deviated action being determined if at least one of the following event occurs:
a. one or more medical instruments used in the virtual action are different from those in the corresponding expected action; and/or
b. the sequence of virtual actions is different from the sequence of expected actions; and/or
c. the time duration of at least one virtual action is longer or shorter than a reference time duration for the corresponding expected action; and/or
d. the number of virtual actions is different from the number of expected actions; and/or
e. one or more medical instruments or objects in the virtual or real operating room are positioned in a different place from an expected positioning place; and/or
f. the virtual patient is positioned by the user in a different position from an expected patient position; and/or
g. the difference between the action data indicative of a virtual action and the information data of the corresponding expected action is outside a tolerance range.

3. The method according to claim 1, further comprising:
a. assigning a relevance weight to each deviated action before determining the final performance index; and/or
b. assigning a relevance weight to each deviated action based on the competence level of the user.

4. The method according to claim 1, further comprising:
modifying or confirming the competence level of the user based on the initial competence level and the final performance index.

5. The method according to claim 4, further comprising:
a. modifying the consecutive expected actions if the final performance index is above a final performance threshold and the competence level of the user has been changed compared to the initial competence level; and/or
b. modifying the consecutive expected actions if the final performance index is above a final performance threshold, the competence level of the user has been confirmed compared to the initial competence level, and the training procedure has been repeated by the user more than one time.

6. The method according to claim 4, further comprising:
saving a user history of the virtual actions if the competence level of the user has been changed compared to the initial competence level, wherein the expected pathway of the training procedure is confirmed or modified based also on the user history.

7. The method according to claim 1, further comprising:
generating a final alert signal by an alert module if the final performance index is below a final performance threshold.

8. The method according to claim 1, further comprising, based on the initial competence level of the user:
a. modifying the training procedure by adding the deviated action in the expected pathway of the training procedure as alternative to the corresponding expected action; and/or
b. modifying the training procedure by replacing an expected action with a corresponding deviated action in the expected pathway of the training procedure.

9. The method according to claim 1, further comprising,
determining at least an intermediate performance index of the user based at least on the number of deviated actions occurred during the training procedure up to an intermediate evaluation point of the expected pathway; and
assigning a different specific competence level or confirming the competence level to the user for the remaining duration of the training procedure based on a comparison between said intermediate performance index and an intermediate performance threshold.

10. The method according to claim 1, wherein the plurality of competence levels comprise at least a first level, a second level, and a third level based on at least the experience of the user in carrying out a real medical procedure and on the number of repeated virtual reality medical training procedures.

11. The method according to claim 1, further comprising:
providing the user with an alert message each time a deviated action is determined during the training procedure.

12. The method according to claim 1, wherein the expected pathway of the training procedure has at least a complexity degree based on the initial specific competence level assigned to the user.

13. A computer program product comprising computer readable instructions which, when implemented on a computer or a control unit, causes the computer to carry out a method according to claim 1.

14. A storage medium comprising the computer program according to claim 13.

15. A workstation for carrying out a medical training procedure, comprising:
a computer system implementing computer readable instructions of the computer program product according to claim 13;
a head mounted device, in particular a virtual reality headset; and
a haptic device.
